SEQUENCE LISTING (4 sequences)

SEQ ID NO: 1 - protein, 123 aa, Mus musculus
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNE

SEQ ID NO: 2 - protein, 323 aa, Mus musculus
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - DNA, 369 bp, Homo sapiens
  source 1..369: mol_type = genomic DNA
cagctgctgttcaacaagaccaagagcgtggagttcaccttttgcaatgacacagtggtcatcccttgttttgtgacaaacatggaggcccagaataccacagaggtgtacgtgaagtggaagttcaagggcagggacatctatacctttgatggcgccctgaacaagtccaccgtgcctacagacttcagctccgccaagatcgaggtgagccagctgctgaagggcgatgcctccctgaagatggacaagagcgatgccgtgtcccacaccggcaattacacatgcgaggtgaccgagctgacacgggagggagagaccatcatcgagctgaagtatagagtggtgtcttggtttagccctaacgag

SEQ ID NO: 4 - DNA, 972 bp, Homo sapiens
  source 1..972: mol_type = genomic DNA
atgtggccactggtggccgccctgctgctgggctctgcctgctgtggcagcgcccagctgctgttcaacaagaccaagagcgtggagttcaccttttgcaatgacacagtggtcatcccttgttttgtgacaaacatggaggcccagaataccacagaggtgtacgtgaagtggaagttcaagggcagggacatctatacctttgatggcgccctgaacaagtccaccgtgcctacagacttcagctccgccaagatcgaggtgagccagctgctgaagggcgatgcctccctgaagatggacaagagcgatgccgtgtcccacaccggcaattacacatgcgaggtgaccgagctgacacgggagggagagaccatcatcgagctgaagtatagagtggtgtcttggtttagccctaacgagaatatcctgatcgtgatcttcccaatctttgccatcctgctgttctggggccagtttggcatcaagacactgaagtaccgctctggcggcatggatgagaagaccatcgccctgctggtggcaggactggtcatcacagtgatcgtgatcgtgggcgccatcctgttcgtgccaggcgagtattctctgaagaacgcaaccggactgggactgatcgtgaccagcacaggcatcctgatcctgctgcactactacgtgttcagcaccgccatcggcctgacatcttttgtgatcgccatcctggtcatccaggtcatcgcctacatcctggcagtggtgggactgtccctgtgcatcgcagcatgtatcccaatgcacggaccactgctgatctccggactgtctatcctggccctggcacagctgctgggactggtgtatatgaagttcgtggcctccaatcagaagacaatccagccacctaggaaggcagtggaggagcccctgaacgcctttaaggagtctaagggcatgatgaatgacgagtga

(19) United States
(12) Patent Application Publication
(10) Pub. No.: US 2023/0295661 A1
(43) Pub. Date: Sep. 21, 2023

(54) PERSISTENT HSV GENE DELIVERY SYSTEM

(71) Applicant: University of Houston System, Houston, TX (US)

(72) Inventors: Shaun ZHANG, Houston, TX (US); Xinping FU, Houston, TX (US)

(21) Appl. No.: 18/184,568

(22) Filed: Mar. 15, 2023

Related U.S. Application Data
(60) Provisional application No. 63/269,440, filed on Mar. 16, 2022.

(51) Int. Cl. C12N 15/86; C07K 14/705; A61K 35/763; A61K 9/00; A61P 25/00

(52) U.S. Cl. CPC C12N 15/86; C07K 14/70596; A61K 35/763; A61K 9/0019; A61P 25/00

(57) ABSTRACT

This invention relates to herpes simplex virus (HSV) based vectors for delivering transgenes (e.g., a therapeutic gene) which are more resistant to neutralization, phagocytosis, and NK cells by immune systems, and methods for their preparation and treatment of disorders and diseases (such as those related to gene expression) with them. In one embodiment, the HSV vectors are prepared by treatment in immune sera that contain a high level of anti-HSV antibodies. The HSV vectors may include an extracellular CD47 domain inserted into the N-terminus of a glycoprotein in order to inhibit phagocyte activity, and the absence of gE for evading NK cells.

This application claims the benefit of U.S. Provisional Application No. 63/269,440, filed Mar. 16, 2022, which is hereby incorporated by reference.

### SEQUENCE LISTING

The instant application contains a Sequence Listing which has been submitted electronically in XML format and is hereby incorporated by reference in its entirety. Said XML copy, created on Apr. 20, 2023, is named 312170-04501_SL.xml and is 6,230 bytes in size.

### FIELD OF THE INVENTION

This invention relates to non-replicating herpes simplex virus (HSV) based gene delivery vectors for delivering transgenes (e.g., a therapeutic gene) which are more resistant to neutralization and phagocytosis by immune systems, and methods for their preparation and treatment of disorders and diseases (such as those related to gene expression) with them.

### BACKGROUND OF THE INVENTION

Gene therapy has many potential clinical applications for treating a variety of diseases such as genetic diseases, autoimmune diseases, and cancer. The success of gene therapy heavily relies on the availability of gene delivery vectors, such as viral vectors. An ideal gene delivery vector can deliver the therapeutic genes efficiently to body tissues and ensure that the delivered therapeutic genes can stay in the tissue long-term. The efficacy of viral vectors, however, is often diminished by the host's various defense mechanisms in the body.

For instance, anti-HSV neutralizing antibodies bind to introduced viral particles, such as herpes simplex virus (HSV) particles. These antibodies can be either pre-existing due to the patient's previous exposure to the virus or freshly developed from repeated administration of the virus during treatment. The neutralizing antibodies can diminish gene transuduction efficiency by several mechanisms. First, they prevent the virus from infecting cells. Second, they can bind to the transduced cells, leading to their destruction either by the host's antibody-dependent cellular phagocytosis (ADCP) or by activation of complement system for the formation membrane attack complex (MAC) (Huber et al., 2001; Tay, Wiehe, and Pollara, 2019; Xie, Jane-Wit, and Pober, 2020). Indeed, animal studies have shown that the pre-existing humoral immunity is detrimental to the infectivity of HSVs (Fu and Zhang, 2001).

The anti-HSV neutralizing antibodies are found to be mainly targeted at two of the virus-encoded glycoproteins—glycoprotein D (gD) and gB (Cairns et al., 2015; Cairns et al., 2014). Both gD and gB contain many neutralizing epitopes, presenting at diverse orders of immunodominant hierarchy in different individuals (Bender et al., 2007). Overall, these epitopes are well-conserved among infected individuals and/or different species (Eing, Kuhn, and Braun, 1989). The studies have also indicated that HSV-2 is more difficult to neutralize than HSV-1 (Silke Heilingloh et al., 2020). The presence of strong epitopes presents as a major obstacle for systemic administration of HSV-based viruses as neutralizing antibodies can readily recognize them and abolish their therapeutic activity.

Another important limiting factor that reduces the effectiveness of systemically delivered viral vectors is the host's mononuclear phagocyte system (MPS). It has been shown that, after systemic delivery, the viral particles can be quickly cleared by MPS (Ellermann-Eriksen, 2005; Hume, 2006; Van Strijp et al., 1989). Specifically, studies by Fulci et al. have shown that depletion of macrophages can profoundly improve the tissue distribution of HSV (Fulci et al., 2007).

Yet another significant antiviral mechanism that can reduce the effectiveness of viral delivery with HSV is natural killer (NK) cells (Alvarez-Breckenridge et al., 2012a; Alvarez-Breckenridge et al., 2012b). The viral glycoprotein E (gE) is known to bind to IgG (Dubin et al., 1994). Recent studies have shown that NK cells can recognize HSV or HSV infected cells via the binding of the surface CD16 activating receptor with the Fc region in IgG bound to gE (Dai and Caligiuri, 2018).

One way to limit the effect of neutralizing antibodies on the infectivity of viruses is by mutagenizing each of the major epitopes. Indeed, a recent study has shown that mutagenizing two of these epitopes from the gD of an HSV can indeed abolish the neutralization by the corresponding monoclonal antibodies (mAbs) (Tuzmen et al., 2020). However, both gB and gD of HSV-1 and HSV-2 are the major targets of the neutralizing antibodies and each of them contain almost a dozen of neutralizing epitopes. Using a traditional way of mutagenizing each of these epitopes from two glycoproteins in the context of a viral genome of over 150 kb long is not practical. Moreover, this approach only deals with one of three major obstacles facing systemic delivery of virotherapy—the neutralizing antibodies, and leaves the MPS untouched.

U.S. Pat. Pub. No. 2012/0301506 and U.S. Pat. Nos. 8,986,672 and 10,039,796 disclose modified HSV-2 and their usage in treating malignant tumors.

Fu et al., Oncotarget, 2018, 9(77):34543-34553, describes genetically engrafting CD47 to the membrane envelop of an HSV to enable it to escape from the MPS.

U.S. Patent Publication No. 2019/0267845 describes a HSV vector that does not express HSV genes in non-complementing cells and which comprises a genome comprising one or more transgenes, wherein the vector is capable of expression of a transgene for at least 28 days in non-complementing cells.

U.S. Patent Publication No. 2018/0148711 discloses HSV-based genome editing vectors.

Verlengia et al., Sci Rep., 2017 May 4, 7(1):1507, doi: 10.1038/s41598-017-01635-1, describes engineered HSV vectors for transgene expression in the central nervous system.

There remains a need for persistent viral vectors suitable for gene therapy that can resist the limiting effect from a host's innate and acquired antiviral immune mechanisms.

### SUMMARY OF THE INVENTION

The present inventors discovered that HSV vectors can deliver transgenes more efficiently and the delivered transgene could persist longer by passaging the HSV vectors with immune sera having elevated levels of anti-HSV antibodies and/or by including an extracellular CD47 domain (such as an extracellular human CD47 domain) in the membrane envelope. It was unexpectedly found that passaging of CD47-containing virus resulted in the near complete absence of gE from the viral particles. This allows the virus to escape from the NK cell-mediated antiviral mechanism. The insertion of the extracellular domain of CD47 into the N-terminus of gC was also found to enable the virus to escape neutralization by anti-HSV antibodies, even without passaging. The HSV vectors described herein can be non-oncolytic and are non-toxic.

One embodiment of the present invention is a composition comprising a non-replicating herpes simplex virus (HSV) based gene delivery vector, wherein (i) the HSV-based vector comprises a transgene, and (ii) the HSV-based vector is prepared by passaging at least twice the HSV-based vector with immune sera having elevated levels of anti-HSV antibodies. In one embodiment, the passaging in the immune sera mutates neutralizing epitopes on glycoprotein B and glycoprotein D of the HSV-based vector. The vector preferably also includes a promoter operably linked to the transgene. In one embodiment, the HSV-based vector is non-oncolytic. In another embodiment, the HSV-based vector is persistent.

The HSV-based vector can be HSV-1 or HSV-2. A preferred HSV-based vector is HSV-2.

The HSV-based vector can be an amplicon packaged into a HSV-1 or HSV-2 particle.

In one embodiment, the HSV-based vector has a membrane envelope comprising glycoproteins, wherein at least one of the glycoproteins comprises an extracellular CD47 domain inserted into the N-terminus of a glycoprotein. The glycoprotein may be selected from glycoprotein C, glycoprotein B, glycoprotein D, glycoprotein H, and glycoprotein L. In one preferred embodiment, the membrane envelope includes a glycoprotein C having an extracellular CD47 domain inserted into its N-terminus. The extracellular CD47 domain can be murine derived or human derived. The extracellular CD47 domain can comprise amino acids 19-141 of murine CD47 (SEQ ID NO:1). Alternatively, the extracellular CD47 domain can comprise amino acids 55-423 of human CD47 (SEQ ID NO:3). The vector includes a genome encoding the CD47 domain.

In another embodiment, the HSV-based vector is free or substantially free of gE.

The immune sera can be a mixture comprising rat sera and human sera that has elevated levels of anti-HSV antibodies.

In one embodiment, the HSV-based vector is prepared by passaging at least two times (e.g., at least five times, or seven times) the HSV-based vector in the presence of rat sera having an elevated level of anti-HSV antibodies followed by passaging at least two times (e.g., at least five or ten times, or seventeen or twenty-three times) in the presence of a mixture of rat sera and at least one human serum having an elevated level of anti-HSV antibodies.

In one embodiment, the composition comprises cerebrospinal fluid.

Another embodiment is a non-replicating HSV-based vector comprising a transgene, where the HSV-based vector has a membrane envelope comprising glycoproteins, wherein at least one of the glycoproteins comprises an extracellular CD47 domain inserted into the N-terminus of a glycoprotein. In one embodiment, the HSV-based vector has a membrane envelope comprising glycoproteins, wherein at least one of the glycoproteins comprises an extracellular CD47 domain inserted into the N-terminus of a glycoprotein. The glycoprotein may be selected from glycoprotein C, glycoprotein B, glycoprotein D, glycoprotein H, and glycoprotein L. In one preferred embodiment, the membrane envelope includes a glycoprotein C having an extracellular CD47 domain inserted into its N-terminus. The extracellular CD47 domain can comprise amino acids 19-141 of CD47 (SEQ ID NO:1). Alternatively, the extracellular CD47 domain can comprise amino acids 55-423 of human CD47 (SEQ ID NO:3). In another embodiment, the HSV-based vector is free or substantially free of gE. In one preferred embodiment, the neutralizing epitopes on glycoprotein B and glycoprotein D of the HSV-based vector are mutated. The vector preferably also includes a promoter operably linked to the transgene. In one embodiment, the HSV-based vector is non-oncolytic. In another embodiment, the HSV-based vector is persistent. The vector includes a genome encoding the extracellular CD47 domain (e.g., which may be murine or human derived).

Yet another embodiment is a method of preparing a composition comprising a non-replicating herpes simplex virus (HSV)-based vector (such as an HSV-based vector described herein) comprising passaging at least twice the HSV-based vector comprising a transgene with immune sera that has elevated levels of anti-HSV antibodies. In one embodiment, the immune sera comprises mammalian anti-HSV antibodies. In another embodiment, the immune sera is a mixture comprising rat sera and human sera that has elevated levels of anti-HSV antibodies. In yet another embodiment, the method comprises passaging the HSV-based vector at least two times (e.g., at least five times, or seven times) in the presence of rat sera having an elevated level of anti-HSV antibodies followed by passaging at least once (e.g., at least two, five, or ten times, or seventeen or twenty-three times) in the presence of a mixture of rat sera and at least one human serum having an elevated level of anti-HSV antibodies.

Yet another embodiment is a method of treating a disease in a patient in need thereof comprising administering to the patient a composition described herein or a non-replicating HSV-based vector described herein. The disease or disorder can be a neurological disease or disorder. In one embodiment, the disease or disorder is not an oncologic disease or disorder (e.g., the disease or disorder is not a cancer). The composition or HSV-based vector can be systemically, intramuscularly, intrademally, subcutaneously, or parenterally administered or administered by intracerebroventricular or intraperitoneal injection. In one embodiment, the patient has been exposed to or vaccinated against HSV-1 and/or HSV-2, or has HSV-1 and/or HSV-2.

Yet another embodiment is a method of vaccinating a patient against a malignant or infectious disease comprising administering to the patient a composition described herein or a non-replicating HSV-based vector described herein. In one embodiment, the transgene is one useful for vaccinating a patient.

### BRIEF DESCRIPTION OF THE DRAWINGS

For a more complete understanding of the present invention, including features and advantages, reference is now made to the detailed description of the invention along with the accompanying figures:

FIG. 1 is a bar chart showing differential sensitivity of trained and untrained FusOn-H2 to anti-HSV sera. 1×10⁴ untrained Fuson-H2 or trained FusOn-SS9 were mixed with either human or rat anti-HSV-2 sera at a final concentration of 1:5. After 1 h incubation at 37° C., the solution was applied to Vero monolayer at a 12-well plate. The plaques were countered 48 h later after crystal violet staining.

FIG. 2A is a schematic illustration of inserting the CD47 encoding sequence into the HSV vector. It shows the insertion of an EGFP-Luc gene cassette (the transgene, which could be any therapeutic gene) and the CMVp-HAtag-CD47ECD into the backbone of an HSV vector (FusOn-H2) which was done through homologous recombination, with the left flanking (LF) sequence and the 3′ region of gC (from the transmembrane domain to the polyA) as the right flanking sequence. The details of individual components in the gene cassette are depicted in the drawings and are labelled accordingly. The abbreviations are: CMVp, cytomegalovirus immediate early promoter; LTR, Rous Sarcoma long terminal repeats that contain the promoter region of this virus; GFP-Luc, EGFP-luciferase fusion gene; HA, HA tag; CD47, murine CD47 extracellular domain; gC, the complete gC coding region. LF, the left flanking region of gC. The recombinant viruses were identified by GFP expression and purified to homogeneity.

FIG. 2B is a schematic illustration of FusOn-Luc construction strategy. It shows FusOn-Luc, which contains the transgene only without CD47 (as a control virus), was constructed in a similar way, except that it does not contain the CD47 extracellular domain.

FIG. 3 is a chart showing the comparison of FusOn-CD47 and FusOn-Luc for ability to transduce cells and to replicate in the presence of phagocytes. It is a comparison of virus yield with or without the presence of phagocytes. Vero cells in 12-well plates were infected with either virus at 0.1 pfu/cell without or with the presence of 200,000 splenocytes. Cells were harvested 48 h and the virus yield was determined by plaque assay. ★p<0.05 as compared with other three wells.

FIG. 4A is a chart showing the mean photon reading (i.e., the quantifty of Luc transgene expression) from daily IVIS imaging measurement, demonstrating that FusOn-CD47 can be more efficiently delivered and the transgene persists longer than FusOn-Luc by the systemic route of delivery (in this case, to the tumor tissues). The tumor was established at the right flank of Balb/c mice by subcutaneous implantation of CT26 cells. Once tumor reached the approximate size of 8 mm in diameter, 2×10⁶ pfu of either FusOn-CD47 or FusOn-Luc was given systemically.

FIG. 4B is a time-lapse of images of the transgene expression in typical mice days after systemic delivery of FusOn-CD47. It shows that the transgene can be more efficiently delivered by FusOn-CD47 than FusOn-Luc by the systemic route. Tumor was established at the right flank of Balb/c mice by subcutaneous implantation of CT26 cells. Once tumor reached the approximate size of 8 mm in diameter, 2×10⁶ pfu of either FusOn-CD47 or FusOn-Luc was given systemically. Animals were imaged daily starting on day 2 for luciferase expression.

FIG. 5 is a chart showing enhanced resistance of FusOn-CD47 to the neutralization effect by the anti-HSV sera after training. 1×10⁴ untrained FusOn-CD47 or the trained FusOn-CD47-SS24 (7 passages in the presence of rat sera and 17 passages in the presence of rat sera plus one human serum) were mixed with either human or rat anti-HSV-2 sera at a final concentration of 1:5. After 1 h incubation at 37° C., the solution was applied to Vero monolayer at a 12-well plate. The plaques were countered 48 h later after crystal violet staining.

FIG. 6 is a chart showing trained FusOn-CD47 acquired additional resistance to anti-HSV immune sera. 5×10³ untrained Fuson-CD47 or the same virus trained at different stage [serial selection 24 (FusOn-CD47-SS24), serial selection 49 (HR49, N2-N5 and N7-N8)] were mixed with an eight-human sera mixture at different dilutions. The trained FusOn-H2 (FusOn-SS9) was also included in this experiment. After 1 h incubation at 37° C., the solution was applied to Vero monolayer at a 12-well plate. The plaques were countered 48 h later after crystal violet staining.

FIG. 7 is a bar chart showing that the insertion of CD47 can enhance the ability of the HSV vector to escape neutralizing antibodies. Five hundred pfu of FusOn-CD47 and FusOn-Luc were incubated with anti-HSV-2 sera at the indicated dilution (1:40 or 1:160) at 37° C. for 1 hour before they were used to infect Vero cells in a 6-well plate. The same number of viruses was incubated with medium only as a control. The cells were stained with crystal violet 48 hours later for enumeration of viral plaques. The percentage of plaques was calculated by dividing the plaque number in the wells containing anti-HSV sera with those from the wells with the same virus without the anti-HSV sera (the controls). ★ indicates a p<0.05 as compared with FusOn-Luc.

FIG. 8 is a time-lapse of images on the transgene expression in typical mice days after systemic delivery of FusOn-CD47, FusOn-Luc or FusOn-SD. It shows that FusOn-CD47, but not FusOn-Luc, could be sufficiently delivered by the systemic route, to mice that have pre-existing anti-HSV immunity, and the passaging of FusOn-CD47 in sera containing anti-HSV antibodies (to generate FusOn-SD) further enhanced the ability of the virus to be delivered by systemically in these mice. Balb/c mice were vaccinated with FusOn-H2 and then implanted with CT26 tumor cells at the right flank. Mice in separate groups were given with each of these three viruses via the tail vein at the dose of 1×10⁷ pfu. Mice were imaged using an IVIS imager at the indicated days after virus injection.

FIG. 9A is a bar chart showing the enzyme-linked immunosorbent assay (ELISA) on gE expression (via the absorbance at 450 nm) on FusOn-H2, FusOn-SD, and PBS (negative control) after incubating the viruses initially with mouse anti-HSV-2 gE (1:1000 dilution) and then with HPR-conjugated rabbit anti-mouse IgG (1:10,000 dilution).

FIG. 9B is a Western blot showing the absence of gE in FusOn-SD but not in FusOn-H2. The viruses were first incubated with mouse anti-HSV-2 gE (1:1000 dilution) and then with HPR-conjugated rabbit anti-mouse IgG (1:10,000 dilution) for the Western blot detection.

FIG. 9C is a schematic illustration on the mechanism of NK cell recognition of HSV or HSV infected cells via gE.

### DETAILED DESCRIPTION OF THE INVENTION

### Introduction

Gene delivery vectors based on herpes simplex viruses (HSVs) have some unique features that are appealing for certain clinical applications. For example, as the HSV genome can stay in neuronal tissues life-long, HSV-based gene delivery vectors can thus be particularly useful for gene therapy for neurological disorders. However, as they are virus-based vectors, the host's antiviral immune mechanisms can often clear the viral vectors, thus limiting their efficiency in gene delivery. Both the host's innate and acquired antiviral immunity can clear HSV-based vectors. The major innate antiviral immune components are macrophages and natural killer cells (NK cells), and the main acquired antiviral immune component is the neutralizing antibodies. Here we report our invention on a series of modifications on HSV-based gene delivery vectors that enable the virus to escape these antiviral mechanisms for an enhanced gene delivery efficiency. For the phagocytosis by macrophages, the extracellular domain of CD47, a molecule that contains a “don't eat me” signal, was inserted into the N-terminus of the glycoprotein C (gC). The modification coats the viral vectors with CD47, which prevents macrophages and other phagocytes from “eating” the viral vectors. For the neutralizing antibodies, the viral vectors were subjected to a series of passages in the presence of anti-HSV immune sera. This not only selected vector candidates with the ability to withstand the neutralizing antibodies but also minimized the gE incorporation into the viral particle. As gE can bind to IgG, which can be subsequently recognized by NK cells to kill cells transduced by HSV-based vectors, the absence of gE from the viral particles allows more efficient gene transduction by enabling the cells to escape from NK cell-mediated killing effect.

Due to the unique property of long-term presence in the neural tissues, HSV-based gene delivery vectors are particularly useful for gene therapy applications in treating neurological disorders. However, both innate and acquired antiviral mechanisms are major hurdles for HSV-based gene delivery vectors, comprising their gene delivery efficiency. The invention disclosed here addresses these hurdles, allowing the viral vectors to be used more effectively, exemplarily, for their clinical application for gene therapy.

### Definitions

The term “Herpes Simplex Virus” or “HSV” as used herein refers to an enveloped, icosahedral, double-stranded DNA virus that infects mammals, including humans. Wild-type HSV infects and replicates in both terminally differentiated non-dividing cells and dividing cells. The HSV vector can be a HSV-based genome editing vector, as described in U.S. Patent Publication No. 2018/0148711. The HSV vector can also be a HSV vector that does not express toxic HSV genes in non-complementing cells and which comprises a genome comprising one or more transgenes as described in U.S. Patent Publication No. 2019/0276845.

The term “HSV-2 persistent virus” and “HSV-2 mutant” are used interchangeably herein.

The term “cell membrane fusion” and “fusion” as used herein refers to fusion of an outer membrane of at least two cells, such as two adjacent cells, for example.

The term “enhanced fusogenic activity” as used herein refers to an enhancement, increase, intensification, argumentation, amplification, or combination thereof of the cell membrane fusion.

The term “persistent” as used herein refers to a viral infection characterized as those in which the virus is not cleared but remains in specific cells of infected individuals. Persistent infections may involve stages of both silent and productive infection without rapidly killing or even producing damage to the host cells.

The term “vector” as used herein refers to a carrier nucleic acid molecule into which a nucleic acid sequence can be inserted for introduction into a cell where it can be replicated. The inserted nucleic acid sequence is referred to as “exogenous” either when it is foreign to the cell into which the vector is introduced or when it is homologous to a sequence in the cell but in a position within the host cell nucleic acid in which die sequence is ordinarily not found. Viral vectors are encapsulated in viral proteins and capable of infecting cells. A vector can be constructed standard recombinant techniques. Generally, these include Sambrook et al., Molecular Cloning: A Laboratory Manual, 2nd Ed., Cold Spring Harbor Laboratory Press (1989) and the references cited therein. Virological considerations are also reviewed in Coen D. M, Molecular Genetics of Animal Viruses in Virology, 2nd Edition, B. N. Fields (editor), Raven Press, N.Y. (1990) and the references cited therein.

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

A “promoter” is a control sequence that is a region of a nucleic acid sequence at which initiation and rate of transcription are controlled. It may contain genetic elements at which regulatory proteins and molecules may bind, such as RNA polymerase and other transcription factors to initiate or regulate the temporal and spatial transcription of a nucleic acid sequence. The phrases “operatively positioned,” “operably linked,” “under control,” and “under transcriptional control” mean that a promoter is in a correct functional location and/or orientation in relation to a nucleic acid sequence to control transcriptional initiation and/or expression of that sequence. Exemplary non-limiting promoters include: a constitutive promoter, a tissue-specific promoter, a tumor-specific promoter, or an endogenous promoter under the control of an exogenous inducible element.

The term “constitutive promoter” as used herein refers to a promoter that drives expression of a gene or polynucleotide in a continuous temporal manner throughout the cell cycle. A constitutive promoter may be cell or tissue-type specific as long as it operates in a continuous fashion throughout the cell cycle to drive the expression of the gene or polynucleotide with which it is associated. Exemplary non-limiting constitutive promoters include: the immediate early cytomegalovirus (CMV) promoter, SV40 early promoter, RSV LTR, Beta chicken actin promoter, and HSV TK promoter.

The term “enhancer” refers to a cis-acting regulatory sequence involved in the control of transcriptional activation of a nucleic acid sequence.

The term “effective” or “therapeutically effective” as used herein refers to suppressing or inhibiting an exacerbation in symptoms, inhibiting, suppressing, or preventing onset of a disease, inhibiting, suppressing, or preventing spread of disease, amelioration of at least one symptom of disease, or a combination thereof.

The term “patient” or “subject” refers to a mammal such as human or a domestic animal (e.g., a dog or cat). In a preferred embodiment, the patient or subject is a human.

The term “sera” or “serum” refers to the fluid from blood that remains when hematocytes and clotting proteins are removed.

The phrases “pharmaceutically” or “pharmacologically acceptable” as used herein refer to molecular entities and compositions that do not produce an adverse, allergic or other untoward reaction when administered to an animal, or human, as appropriate. The phrase “pharmaceutically acceptable carrier” includes any and all solvents, dispersion media, coatings, antibacterial and antifungal agents, isotonic and absorption delaying agents and the like.

The term “unit dose” refers to a physically discrete unit suitable for use in a subject, each unit containing a predetermined quantity of the therapeutic composition calculated to produce the desired response in association with its administration, i.e., the appropriate route and treatment regimen.

The invention may be utilized as a stand-alone therapy or in conjunction with another means of therapy, including pharmaceutical treatments and surgery.

### Vector Construction

The HSV vector comprises a transgene encoding a desired protein and in specific embodiments further comprises a regulatory sequence, such as a promoter. The recombinant virus can be further comprised of some or all of the following components.

Vectors

Vectors, include but are not limited to, plasmids, cosmids, viruses (bacteriophage, animal viruses, and plant viruses), and artificial chromosomes (e.g., YACs). Methods for the construction of engineered viruses and DNA vectors are known in the art. Generally these include Sambrook et al., Molecular Cloning: A Laboratory Manual, 2nd Ed., Cold Spring Harbor Laboratory Press (1989) and the references cited therein. Virological considerations are also reviewed in Coen D. M, Molecular Genetics of Animal Viruses in Virology, 2^{nd} Edition, B. N. Fields (editor), Raven Press, N.Y. (1990) and the references cited therein.

Expression vectors can contain a variety of “control sequences,” which refer to nucleic acid sequences necessary for the transcription and possibly translation of an operably linked coding sequence (such as a transgene) in a particular host cell. In addition to control sequences that govern transcription and translation, DNA vectors, expression vectors, and viruses may contain nucleic acid sequences that serve other functions as well.

In one embodiment, the HSV-based vector does not express ICP0, ICP4, ICP22, and ICP27 as immediate early genes. Such vectors can be prepared as described in U.S. Patent Publication No. 2019/0276845, which is hereby incorporated by reference.

The HSV vector can be an amplicon packaged into HSV-1 or HSV-2 particles. HSV amplicons are unique gene delivery vectors. They are plasmid-based constructs that contain, in addition to the therapeutic gene(s), a copy of the HSV replication origin and a packaging signal. When co-delivered into cells together with a HSV virus as a helper, the amplicon constructs will be replicated (due to the presence of the replication origin) and then the replicated amplicon sequences will be packaged into viral particles (due to the presence of the packaging signal). The packaged amplicon possesses the same gene transduction property as the helper virus. All the HSV viruses described in this disclosure can be applied as a helper virus for HSV amplicon packaing for gene delivery.

1. Promoters and Enhancers

A promoter generally comprises a sequence that functions to position the start site for RNA synthesis. The best-known example of this is the TATA box, but in some promoters lacking a TATA box (e.g., the promoter for the mammalian terminal deoxynucleotidyl transferase gene and the promoter for the SV40 late genes) a discrete element overlying the start site itself helps to fix the place of initiation. Additional promoter elements regulate the frequency of transcriptional initiation. Typically, these are located in the region 30 to 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an enhancer.

A promoter may be one naturally associated with a nucleic acid sequence, as may be obtained by isolating the 5′ non-coding sequences located upstream of the coding segment and/or exon. Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the R lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR, in connection with the compositions disclosed herein (see U.S. Pat. Nos. 4,683,202 and 5,928,906). Furthermore, it is contemplated that control sequences, which direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination may be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art. Non-limiting examples of such regions include the human LIMK2 gene (Nomoto et al. (1999) Gene 236(2):259-271), the somatostatin receptor-2 gene (Kraus et al., (1998) FEBS Lett. 428(3): 165-170), murine epididymal retinoic acid-binding gene (Lareyre et al., (1999) J. Biol. Chem. 274(12):8282-8290), human CD4 (Zhao-Emonet et al., (1998) Biochem. Biophys. Acta, 1442(2-3):109-119), mouse α-2 (XI) collagen (Tsumaki, et al., (1998), J. Biol. Chem. 273(36):22861-4) D1A dopamine receptor gene (Lee, et al., (1997), DNA Cell Biol. 16(11):1267-1275) insulin-like growth factor II (Vu et al., (1997) Biophys Biochem Res. Comm. 233(1):221-226) and human platelet endothelial cell adhesion molecule-1 (Almendro et al., (1996) J. Immunol. 157(12):5411-5421).

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819).

3. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in other embodiments involving eukaryotes, it is contemplated that the terminator comprise a signal for the cleavage of the RNA, and that the terminator signal promote polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

4. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, both of which are convenient and known to function well in various target cells. Polyadenylation may increase the stability of the transcript or may facilitate cytoplasmic transport.

5. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with fluorescence activated cell sorting (FACS) analysis. The marker used is not believed to be important, so long as it is capable of being expressed simultaneously with the nucleic acid encoding a gene product. Further examples of selectable and screenable markers are well known to one of skill in the art.

The vector is introduced to the initially infected cell by suitable methods. Such methods for nucleic acid delivery for transformation of an organelle, a cell, a tissue or an organism for use with the current invention are believed to include virtually any method by which a nucleic acid (e.g., HSV vector) can be introduced into an organelle, a cell, a tissue or an organism, as described herein or as would be known to one of ordinary skill in the art. Non-limiting exemplary methods include: direct delivery of DNA by ex vivo transfection; injection (U.S. Pat. Nos. 5,994,624, 5,981,274, 5,945,100, 5,780,448, 5,736,524, 5,702,932, 5,656,610, 5,589,466 and 5,580,859); microinjection (U.S. Pat. No. 5,789,215); electroporation (U.S. Pat. No. 5,384,253); calcium phosphate precipitation; DEAE dextran followed by polyethylene glycol; direct sonic loading; liposome mediated transfection; receptor-mediated transfection; microprojectile bombardment (PCT Application Nos. WO 94/09699 and 95/06128; U.S. Pat. Nos. 5,610,042; 5,322,783 5,563,055, 5,550,318, 5,538,877 and 5,538,880); agitation with silicon carbide fibers (U.S. Pat. Nos. 5,302,523 and 5,464,765); Agrobacterium mediated transformation (U.S. Pat. Nos. 5,591,616 and 5,563,055); PEG mediated transformation of protoplasts (U.S. Pat. Nos. 4,684,611 and 4,952,500); desiccation/inhibition mediated DNA uptake, and any combination of these methods, or other methods known to persons of skill in the art. The composition can also be delivered to a cell in a mammal by administering it systemically, such as intravenously, in a pharmaceutically acceptable excipient.

Methods of DNA Vector Delivery to Cells

1. Ex Vivo Transdduction

Methods for transfecting cells and tissues removed from an organism in an ex vivo setting are known to those of skill in the art. Thus, it is contemplated in the present invention that cells or tissues may be removed and transfected ex vivo using the nucleic acids and compositions described herein. In particular aspects, the transplanted cells or tissues may be placed into an organism. In some embodiments, a nucleic acid is expressed in the transplanted cell or tissue.

2. Injection

In certain embodiments, a nucleic acid may be delivered to an organelle, a cell, a tissue or an organism via one or more injections (i.e., a needle injection), such as, for example, subcutaneously, intradermally, intramuscularly, intravenously, intraperitoneally, etc. Methods of injection are well known to those of ordinary skill in the art (e.g., injection of a composition comprising a saline solution). Further embodiments of the present invention include the introduction of a nucleic acid by direct microinjection. The amount of composition of the present invention used may vary upon the nature of the cell, tissue or organism affected.

3. Electroporation

In certain embodiments of the present invention, a nucleic acid is introduced into an organelle, a cell, a tissue or an organism via electroporation. Electroporation involves the exposure of a suspension of cells and DNA to a high voltage electric discharge. In some variants of this method, certain cell wall degrading enzymes, such as pectin degrading enzymes, are employed to render the target recipient cells more susceptible to transformation by electroporation than untreated cells (U.S. Pat. No. 5,384,253). Alternatively, recipient cells can be made more susceptible to transformation by mechanical wounding.

4. Liposome Mediated Transfection

In a further embodiment of the invention, a composition as described herein (such as a vector) may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self rearrangement before the formation of closed structures and entrap water and dissolved solutes between the lipid bilayers. Also contemplated is an nucleic acid complexed with Lipofectamine (Gibco BRL) or Superfect (Qiagen).

In certain embodiments of the invention, a liposome may be complexed with a hemagglutinatin virus (HVJ). This has been shown to facilitate fusion with the cell membrane and promote cell entry of liposome encapsulated DNA (Kaneda et al., (1989) Science 20; 243(4889):375-8). In other embodiments, a liposome may be complexed or employed in conjunction with nuclear non histone chromosomal proteins (HMG1) (Kato et al., (1991) J Boil Chem. (1991) February 25; 266(6):3361-4). In yet further embodiments, a liposome may be complexed or employed in conjunction with both HVJ and HMG 1. In other embodiments, a delivery vehicle may comprise a ligand and a liposome.

5. Receptor Mediated Transfection

A nucleic acid may be delivered to a target cell via receptor mediated delivery vehicles. This approach takes advantage of the selective uptake of macromolecules by receptor mediated endocytosis. In view of the cell type specific distribution of various receptors, this delivery method adds another degree of specificity to the present invention.

In certain embodiments, the receptor mediated gene targeting vehicle comprises a receptor specific ligand and a nucleic acid binding agent. Other embodiments comprise a receptor specific ligand to which the nucleic acid to be delivered has been operatively attached. Several ligands have been used for receptor mediated gene transfer including the epidermal growth factor (EGF), which has been used to deliver genes to squamous carcinoma cells as described in European Patent No. EPO 0 273 085.

In other embodiments, a nucleic acid delivery vehicle component of a cell specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell.

In still further embodiments, the nucleic acid delivery vehicle component of a targeted delivery vehicle may be a liposome itself, which will preferably comprise one or more lipids or glycoproteins that direct cell specific binding. For example, lactosyl ceramide, a galactose terminal asialganglioside, has been incorporated into liposomes and an increase in the uptake of the insulin gene by hepatocytes has been observed (Nicolau et al., (1987) Methods Enzymol. 149:157-76). It is contemplated that the tissue specific transforming constructs of the present invention can be specifically delivered into a target cell in a similar manner.

6. Microprojectile Bombardment

Microprojectile bombardment techniques can be used to introduce a nucleic acid into at least one, organelle, cell, tissue or organism (U.S. Pat. Nos. 5,550,318; 5,538,880; 5,610,042; and PCT Application No. WO 94/09699). This method depends on the ability to accelerate microprojectiles that are either coated with DNA or contain DNA, to a high velocity allowing them to pierce cell membranes and enter cells without killing them. The microprojectiles may be comprised of any biologically inert substance, such as tungsten, platinum, or gold. For the bombardment, cells in suspension are concentrated on filters or solid culture medium. Alternatively, immature embryos or other target cells may be arranged on solid culture medium. The cells to be bombarded are positioned at an appropriate distance below the microprojectile bombardment device on a stopping plate. A wide variety of microprojectile bombardment techniques useful for practice with the current invention will be known to persons of skill in the art.

Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells that do not contain a recombinantly introduced nucleic acid.

A tissue may comprise a host cell or cells to be transformed with a cell membrane fusion-generating HSV-2 mutant. The tissue may be part or separated from an organism. In certain embodiments, a tissue may comprise, but is not limited to, adipocytes, alveolar, ameloblasts, neural, basal cells, blood (e.g., lymphocytes), blood vessel, bone, bone marrow, glial cell, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, small intestine, spleen, stem cell, stomach, and testes.

In certain embodiments, the host cell or tissue may be comprised in at least one organism. In certain embodiments, the organism may be, but is not limited to, a prokaryote (e.g., a eubacteria, an archaea) or a eukaryote, as would be understood by one of ordinary skill in the art.

Numerous cell lines and cultures are available for use as a host cell, and are commercially available through organizations such as the American Type Culture Collection (ATCC). An appropriate host can be determined by one of skill in the art based on the vector backbone and the desired result. Exemplary non-limiting cell types available for vector replication and/or expression include bacteria, such as E. coli (e.g., E. coli strains RR1, LE392, B, X 1776 (ATCC No. 31537), W3110, F, lambda, DH5a, JM109, and KC8); bacilli e.g., Bacillus subtilis; other enterobacteriaceae e.g., Salmonella typhimurium, Serratia marcescens, as well as a number of commercially available bacterial hosts and competent cells such as SURE® Competent Cells and SOLOPACK™ Gold Cells (STRATAGENE®, La Jolla, Calif.). Non-limiting examples of eukaryotic host cells for replication and/or expression of a vector include, HeLa, NIH3T3, Jurkat, 293, Cos, CHO, Saos, and PC12.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

Viral Vector Packaging and Propagation

1. Viral Packaging

In specific embodiments of the present invention, the transgene can be inserted into the virus through homologous recombination. Typically, this is done by co-transfecting the plasmid DNA containing the transgene with purified HSV (e.g., HSV-2) genomic DNA into Vero cells using Lipofectamine. The recombinant virus is then identified (typically by screening the virus plaques for the presence of a selectable marker) and selecting plaques containing the transgene. The selected recombinant virus is then characterized in vitro to confirm that the transgene has been correctly inserted into the HSV genome.

2. Preparation of Viral Stocks

Once the recombinant HSV (e.g., HSV-2) mutant virus has been selected, viral stocks can be prepared as follows. Vero cells are grown in 10% fetal bovine serum (FBS) and infected with 0.01 plaque forming units (pfu) per cell. Viruses are then harvested from the cells 2 days later by repeated freezing and thawing and sonication. The harvested virus is then purified as described (Nakamori, et al., (2003) Clinical Cancer Res. 9(7):2727-2733). The purified virus is then titered, aliquoted and stored at −80° C. until use.

Protein Expression Systems

Protein expression systems may be utilized in the generation of DNA vector compositions of the present invention for example, to express the polypeptide encoded by the transgene for functional studies. Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236 and is commercially available (e.g., CLONTECH, Inc. Mountain View, Calif).

Other examples of commercially available expression systems include an inducible mammalian expression system, which involves a synthetic ecdysone-inducible receptor, or a pET expression system, or an E. coli expression system (STRATAGENE, LaJolla, Calif.); A tetracycline-regulated expression system, an inducible mammalian expression system that uses the full-length CMV promoter or a yeast expression system designed for high-level production of recombinant proteins in the methylotrophic yeast Pichia methanolica (INVITROGEN, Carlsbad, Calif.).

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analysis, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

### Pharmaceutical Compositions and Routes of Administration

Compositions of the present invention can be administered as a pharmaceutical composition comprising the modified HSV-based vectors containing one or more copies of therapeutic genes, as described herein. The compositions of the present invention include classic pharmaceutical preparations. In general, the compositions of the present invention can be administered as pharmacological agents by dissolving or dispersing the composition in a pharmaceutically acceptable carrier or aqueous medium. The use of such media and agents for pharmaceutical active substances is well known in the art. Except insofar as any conventional media or agent is incompatible with the compositions of the invention, its use in a therapeutic composition is contemplated. Supplementary active ingredients, such as other anti-disease agents, can also be incorporated into the pharmaceutical composition. Administration of the composition will be via any common route so long as the target cell is available via that route. Exemplary administration routes include oral, nasal, buccal, rectal, vaginal or topical. Alternatively, administration may be by orthotopic, intradermal, subcutaneous, intramuscular, intraperitoneal, intravenous, or direct intratumoral injection. The pharmaceutical formulations, dosages and routes of administration for the compositions of the present invention are described herein. Administration can be by systemic administration or by intracerebroventricular or intraperitoneal injection.

Pharmaceutical Formulation of HSV Vector

The composition or HSV vector of the present invention can be prepared as a pharmacologically acceptable formulation. Typically, the composition or vector is mixed with an excipient which is pharmaceutically acceptable and compatible with the virus. Suitable excipients are, for example, water, saline, dextrose, glycerol, ethanol, or the like and combinations thereof. In addition, if desired, the preparation may contain minor amounts of auxiliary substances such as wetting or emulsifying agents, pH-buffering agents, adjuvants or immunopotentiators, which enhance the effectiveness of the viral mutant (See, Remington's Pharmaceutical Sciences, Gennaro, A. R. et al., eds., Mack Publishing Co., pub., 18th ed., 1990). For example, a typical pharmaceutically acceptable carrier for injection purposes may comprise from 50 mg up to about 100 mg of human serum albumin per milliliter of phosphate buffered saline. Additional non-limiting exemplary non-aqueous solvents suitable for use in the formulation of a pharmacologically acceptable composition include propylene glycol, polyethylene glycol, vegetable oil, sesame oil, peanut oil and injectable organic esters such as ethyloleate. Exemplary non-limiting aqueous carriers include water, aqueous solutions, saline solutions, parenteral vehicles such as sodium chloride, Ringer's dextrose, etc. Intravenous vehicles include fluid and nutrient replenishers. Determining the pH and exact concentration of the various components of the pharmaceutical composition is routine and within the knowledge of one of ordinary skill in the art (See Goodman and Gilman's The Pharmacological Basis for Therapeutics, Gilman, A. G. et al., eds., Pergamon Press, pub., 8th ed., 1990).

Sterile injectable solutions are prepared by incorporating the active compounds in the required amount in the appropriate solvent with various other sterile ingredients as required and described above. Generally, dispersions are prepared by incorporating the various sterilized active ingredients into a sterile vehicle which contains the basic dispersion medium and the required other ingredients as described above.

In certain embodiments, where the invention is used to treat neurological conditions, convection-enhanced delivery (CED) may be utilized. Convection-enhanced delivery (CED) involves the use of fine catheters and precisely controlled infusion rates to distribute therapeutic agents by bulk-flow directly into the brain extracellular space, possibly along the same extracellular pathways that glioma cells are able to migrate. In contrast to techniques of drug delivery that depend on diffusion to achieve adequate drug distribution, such as carmustine-impregnated biodegradable polymers, with CED it is possible to distribute drugs homogeneously over potentially large volumes of brain, irrespective of the molecular size of the therapeutic agent. As such it is an ideal technique for the administration of viral vector-mediated gene therapy to the brain of patients.

In some embodiments, the HSV-based gene delivery vectors may be a composition that further comprises cerebrospinal fluid (CSF), especially artificial cerebrospinal fluid. Artificial cerebrospinal fluid is well known in the art and is a fluid which mimics natural CSF, particularly in terms of its salt contents. Preferably, the composition comprises NaCl at a similar concentration to that found in natural CSF, that is to say the concentration is preferably within 15%, more preferably within 10% of the concentration in natural CSF. Preferably, the composition comprises NaHCO₃ at a similar concentration to that found in natural CSF, that is to say the concentration is preferably within 15%, more preferably within 10% of the concentration in natural CSF. Preferably the composition comprises KCl at a similar concentration to that found in natural CSF, that is to say the concentration is preferably within 15%, more preferably within 10% of the concentration in natural CSF. Preferably, the composition comprises NaH₂PO₄ at a similar concentration to that found in natural CSF, that is to say the concentration is preferably within 15%, more preferably within 10% of the concentration in natural CSF. Preferably, the composition comprises MgCl₂ at a similar concentration to that found in natural CSF, that is to say the concentration is preferably within 15%, more preferably within 10% of the concentration in natural CSF. Preferably the composition comprises glucose at a similar concentration to that found in natural CSF, that is to say the concentration is preferably within 15%, more preferably within 10% of the concentration in natural CSF. Alternatively, the artificial CSF may omit glucose, so as to reduce the likelihood of bacterial growth in any catheter used to administer the composition to a subject.

The composition may further comprise other active agents. Pharmaceutical compositions of this invention may also comprise any pharmaceutically acceptable carrier, adjuvant or vehicle. Pharmaceutically acceptable carriers, adjuvants and vehicles that may be used in the pharmaceutical compositions include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol.

The pharmaceutical compositions of this invention may be administered by any appropriate route, but are preferably administered via injection, especially via a neurocatheter, in particular by convection enhanced delivery. The pharmaceutical compositions may contain any conventional non-toxic pharmaceutically-acceptable carriers, adjuvants or vehicles.

Routes and Dosages for Administration of HSV Vector

The mutant viral composition may be delivered by any route that provides access to the target tissue. Exemplary non-limiting routes of administration may include oral, nasal, buccal, rectal, vaginal topical, or by injection (including orthotopic, intradermal, subcutaneous, intramuscular, intraperitoneal, intravenous, or intracerebroventricular injection). Typically, the viral mutant would be prepared as an injectable, either as a liquid solution or a suspension; a solid form suitable for solution in, or suspension in, liquid prior to injection may also be prepared. The preparation also may be emulsified.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermolysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Those of skill in the art will recognize that the best treatment regimens for using a composition of the present invention to provide therapy can be straightforwardly determined. This is not a question of experimentation, but rather one of optimization, which is routinely conducted in the medical arts. For example, in vivo studies in mice provide a starting point from which to begin to optimize the dosage and delivery regimes. The frequency of injection may initially be once a week. However, this frequency might be optimally adjusted from one day to every two weeks to monthly, depending upon the results obtained from the initial clinical trials and the needs of a particular patient. Human dosage amounts can initially be determined by extrapolating from the amount of composition used in mice.

Dosages

The amount of viral vector delivered will depend on several factors including number of treatments, subject to be treated, capacity of the subjects immune system to synthesize anti-viral antibodies, the target tissue to be destroyed, and the degree of protection desired. The precise amount of viral composition to be administered depends on the judgment of the practitioner and is peculiar to each individual. However, suitable dosage ranges from 10⁵ plaque forming units (pfu) to 10¹⁰ pfu. In certain embodiments, the dosage of viral DNA may be about 10⁵, 10⁶, 10⁷, 10⁸, 10⁹, up to and including 10¹⁰ pfu.

Non-Viral DNA Vector Formulation

In addition to the formulations described above for viral pharmaceutical formulation, the non-viral DNA vector can also be prepared as a sterile powder for the preparation of pharmacologically acceptable sterile solutions. Typical methods for preparation of sterile powder include vacuum-drying and freeze-drying techniques which yield a powder of the active ingredient plus any additional desired ingredient from a previously sterile-filtered solution thereof.

Routes and Dosages for Administration of Non-Viral DNA Vector

Several methods for the delivery of non-viral vectors for the transfer of a polynucleotide of the present invention into a mammalian cell is contemplated. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection as discussed previously. Some of these techniques may be successfully adapted for in vivo or ex vivo use.

In some embodiments of the present invention, the expression vector may simply consist of naked recombinant DNA or plasmids comprising the polynucleotide. Transfer of the construct may be performed by any of the methods mentioned herein which physically or chemically permeabilize the cell membrane. This is particularly applicable for transfer in vitro, but it may be applied to in vivo use as well.

In other embodiments, the delivery vehicle may comprise a ligand and a liposome. For example, Nicolau et al., employed lactosyl-ceramide, a galactose-terminal asialganglioside, incorporated into liposomes and observed an increase in the uptake of the insulin gene by hepatocytes (Nicolau et al., (1987) Methods Enzymol. 149:157-76). Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor (as described in European Patent No. EP 0 273 085) and mannose can be used to target the mannose receptor on liver cells.

In certain embodiments, DNA transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissue.

Dosages

In certain embodiments it is envisioned that the dosage may vary from between about 10³ pfu/kg body weight to about 10⁸ pfu/kg body weight. In certain embodiments, the dosage may be from about 10³, 10⁴, 10⁵, 10⁶, 10⁷, up to and including 10⁸ pfu/kg body weight. Of course, this dosage amount may be adjusted upward or downward, as is routinely done in such treatment protocols, depending on the results of the initial clinical trials and the needs of a particular patient.

### Methods of Treatment

Yet another embodiment is a method of treating a disease in a patient in need thereof comprising administering to the patient a composition described herein or a non-replicating HSV-based vector described herein. The disease or disorder can be a neurological disease or disorder. In one embodiment, the disease or disorder is not an oncologic disease or disorder (e.g., the disease or disorder is not a cancer). The composition or HSV-based vector can be systemically, intramuscularly, intrademally, subcutaneously, or parenterally administered or administered by intracerebroventricular or intraperitoneal injection. In one embodiment, the patient has been exposed to or vaccinated against HSV-1 and/or HSV-2, or has HSV-1 and/or HSV-2.

Yet another embodiment is a method of vaccinating a patient against a malignant or infectious disease comprising administering to the patient a composition described herein or a non-replicating HSV-based vector described herein. In one embodiment, the transgene is one useful for vaccinating a patient.

### Combination Treatments

Other gene therapy can also be used in conjunction with the compositions and methods described herein as a combination therapy for the treatment of a disease or disorder. Gene therapy as a combination treatment relies on the delivery and expression of a therapeutic gene, separate from the HSV vector described herein. The gene therapy can be administered either before, after, or at the same time as the HSV vector described herein. Exemplary non-limiting targets of gene therapy include immunomodulatory agents, agents that affect the up regulation of cell surface receptors and GAP junctions, cytostatic and differentiation agents, or inhibitors of cell adhesion. Exemplary non-limiting immunomodulatory genes that can be used as part of gene therapy in combination with the present invention include tumor necrosis factor; interferon alpha, beta, and gamma; IL-2 and other cytokines; F42K and other cytokine analogs; Or MIP-1, MIP-1 beta, MCP-1, RANTES, and other chemokines.

Hormonal therapy may also be used in conjunction with the present invention. The use of hormones may be employed in the treatment of various diseases to lower the level or block the effects of certain hormones such as testosterone or estrogen.

### EXAMPLES

The following examples are included to demonstrate aspects of the invention. While the examples are shown with respect to an oncolytic virus, those skilled in the art would understand that the beneficial effects of passaging and inclusion of an extracellular CD47 domain also apply to any HSV-based gene delivery vector as described herein.

It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Construction of FusOn-H2

FusOn-H2 was constructed as described in Example 1 of U.S. Pat. No. 10,039,796. To systemically mutate the majority of the neutralizing epitopes on both gB and gD, as well as to enhance the complement antagonizing capability of the gC, FusOn-H2 was subjected to a series of selection in the presence of anti-HSV-2 sera. FusOn-H2 was initially subjected to the selection in cell culture in the presence of a mixture of serum collected from 5 rats that had been vaccinated with HSV and had high anti-HSV antibodies in blood. One of the main reasons to start this selection with rat sera is because they contain both natural immunoglobulins and mannan-binding lectin (MBL) that can activate complement against HSV, while mouse and human sera only contain one of these two activation mechanisms (MBL for mouse and natural immunoglobulins for human, respectively) (Wakimoto et al., 2002). After 7 consecutive rounds of selection under this rat sera mixture, one human serum that contains a high level of anti-HSV-2 antibodies was added into the rat sera and continued the selection.

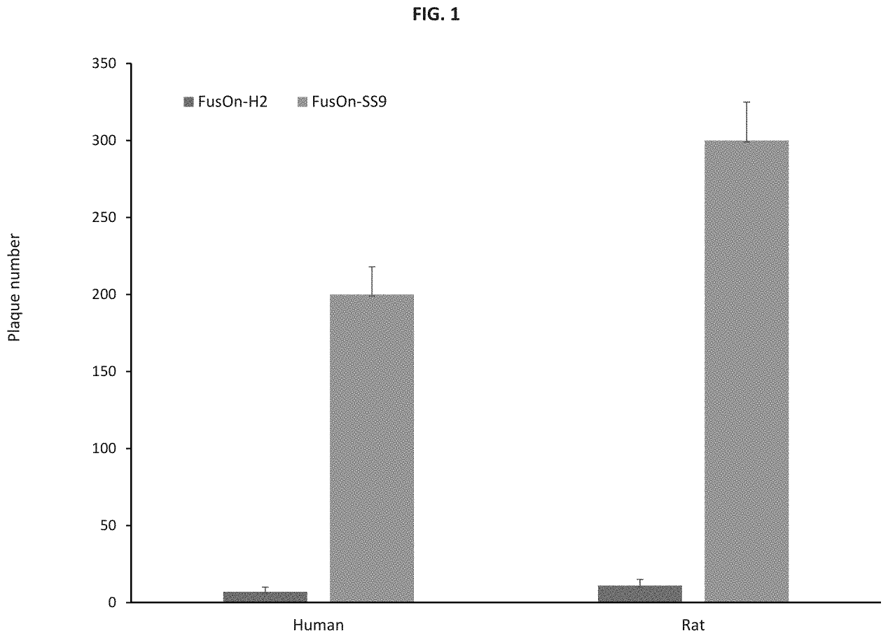

The virus infectivity in the presence of anti-HSV sera was monitored regularly during the selection process and the results indicated the continuous improvement over the unselected FusOn-H2 on the resistance to the neutralizing antibodies. One of the testing results is shown in FIG. 1, which was conducted after the virus underwent a total of 9 rounds of selection (i.e., 7 rounds of selection with the rat sera and 2 rounds of selection with a mixture of rat sera and human serum). The result showed that the trained virus (designated FusOn-559) was more than 28.5- and 27.2-fold more resistant to the neutralizing effect of human and rat anti-HSV-2 sera, respectively.

To enable FusOn-H2 with the ability to escape the host's clearance by the mononuclear phagocyte system (MPS) in the blood during systemic delivery, the possibility of genetically engrafting CD47, a “don't eat me” signal molecule, to the membrane envelope of the virus was explored. Macrophages are the major phagocytes that are responsible for rapid clearance of HSV particles and it has been reported that depletion of macrophages can significantly improve the therapeutic effect of oncolytic HSV (Fulci et al., 2007). The phagocytic activity of macrophages is controlled by both positive and negative regulation mechanisms. Interactions between CD47 and its receptor, SIRPα, provide a strong negative regulation signal (“don't eat me signal”) to macrophages (Kinchen and Ravichandran, 2008).

HSV encodes several glycoproteins that are assembled on the surface of viral envelope. They include glycoprotein C (gC), gB, gD, gH and gL. Each of them can serve as a candidate molecule for incorporating the extracellular domain (ECD) of murine CD47 (mCD47) so that it may be engrafted to the surface of the virus envelope. Glycoprotein C (gC) was chosen here, as unlike other glycoproteins mentioned, it is not essential for virus infectivity. As such, modifying it for incorporating mCD47 would not run the risk of altering the natural tropism of the oncolytic virus. The ECD of mCD47 (aa 19-141) (SEQ ID NO:1) (shown in the table below) was initially inserted into the N-terminus of gC to create the chimeric form of gC (cgC), and its expression is driven by CMV IE promoter. The sequences of the full length murine CD47 (SEQ ID NO:2) and human CD47 (SEQ ID NO:4) are provided in the table below.

| | |
|---|---|
| Amino acids | QLLFNKTKSV EFTFCNDTVV IPCFVTNMEA |
| 19-141 of | QNTTEVYVKW KFKGRDIYTF DGALNKSTVP |
| murine CD47 | TDFSSAKIEV SQLLKGDASL KMDKSDAVSH |
| (SEQ ID NO: 1) | TGNYTCEVTE LTREGETIIE LKYRVVSWFS |
| | PNE |
| Full length | MWPLVAALLL GSACCGSAQL LFNKTKSVEF |
| murine CD47 | TFCNDTVVIP CFVTNMEAQN TTEVYVKWKF |
| (SEQ ID NO: 2) | KGRDIYTFDG ALNKSTVPTD FSSAKIEVSQ |
| | LLKGDASLKM DKSDAVSHTG NYTCEVTELT |
| | REGETIIELK YRVVSWFSPN ENILIVIFPI |
| | FAILLFWGQF GIKTLKYRSG GMDEKTIALL |
| | VAGLVITVIV IVGAILFVPG EYSLKNATGL |
| | GLIVTSTGIL ILLHYYVFST AIGLTSFVIA |
| | ILVIQVIAYI LAVVGLSLCI AACIPMHGPL |
| | LISGLSILAL AQLLGLVYMK FVASNQKTIQ |
| | PPRKAVEEPL NAFKESKGMM NDE |
| Full length | ATGTGGCCAC TGGTGGCCGC CCTGCTGCTG |
| human CD47 | GGCTCTGCCT GCTGTGGCAG CGCCCAGCTG |
| (SEQ ID NO: 4) | CTGTTCAACA AGACCAAGAG CGTGGAGTTC |
| | ACCTTTTGCA ATGACACAGT GGTCATCCCT |
| | TGTTTTGTGA CAAACATGGA GGCCCAGAAT |
| | ACCACAGAGG TGTACGTGAA GTGGAAGTTC |
| | AAGGGCAGGG ACATCTATAC CTTTGATGGC |
| | GCCCTGAACA AGTCCACCGT GCCTACAGAC |
| | TTCAGCTCCG CCAAGATCGA GGTGAGCCAG |
| | CTGCTGAAGG GCGATGCCTC CCTGAAGATG |
| | GACAAGAGCG ATGCCGTGTC CCACACCGGC |
| | AATTACACAT GCGAGGTGAC CGAGCTGACA |
| | CGGGAGGGAG AGACCATCAT CGAGCTGAAG |
| | TATAGAGTGG TGTCTTGGTT TAGCCCTAAC |
| | GAGAATATCC TGATCGTGAT CTTCCCAATC |
| | TTTGCCATCC TGCTGTTCTG GGGCCAGTTT |
| | GGCATCAAGA CACTGAAGTA CCGCTCTGGC |
| | GGCATGGATG AGAAGACCAT CGCCCTGCTG |
| | GTGGCAGGAC TGGTCATCAC AGTGATCGTG |
| | ATCGTGGGCG CCATCCTGTT CGTGCCAGGC |
| | GAGTATTCTC TGAAGAACGC AACCGGACTG |
| | GGACTGATCG TGACCAGCAC AGGCATCCTG |
| | ATCCTGCTGC ACTACTACGT GTTCAGCACC |
| | GCCATCGGCC TGACATCTTT TGTGATCGCC |
| | ATCCTGGTCA TCCAGGTCAT CGCCTACATC |
| | CTGGCAGTGG TGGGACTGTC CCTGTGCATC |
| | GCAGCATGTA TCCCAATGCA CGGACCACTG |
| | CTGATCTCCG GACTGTCTAT CCTGGCCCTG |
| | GCACAGCTGC TGGGACTGGT GTATATGAAG |
| | TTCGTGGCCT CCAATCAGAA GACAATCCAG |
| | CCACCTAGGA AGGCAGTGGA GGAGCCCCTG |
| | AACGCCTTTA AGGAGTCTAA GGGCATGATG |
| | AATGACGAGT GA |
| Amino acids | CAGCTGCTGT TCAACAAGAC CAAGAGCGTG |
| 55-423 of | GAGTTCACCT TTTGCAATGA CACAGTGGTC |
| human CD47 | ATCCCTTGTT TTGTGACAAA CATGGAGGCC |
| (SEQ ID NO: 3) | CAGAATACCA CAGAGGTGTA CGTGAAGTGG |
| | AAGTTCAAGG GCAGGGACAT CTATACCTTT |
| | GATGGCGCCC TGAACAAGTC CACCGTGCCT |
| | ACAGACTTCA GCTCCGCCAA GATCGAGGTG |
| | AGCCAGCTGC TGAAGGGCGA TGCCTCCCTG |
| | AAGATGGACA AGAGCGATGC CGTGTCCCAC |
| | ACCGGCAATT ACACATGCGA GGTGACCGAG |
| | CTGACACGGG AGGGAGAGAC CATCATCGAG |
| | CTGAAGTATA GAGTGGTGTC TTGGTTTAGC |
| | CCTAACGAG |

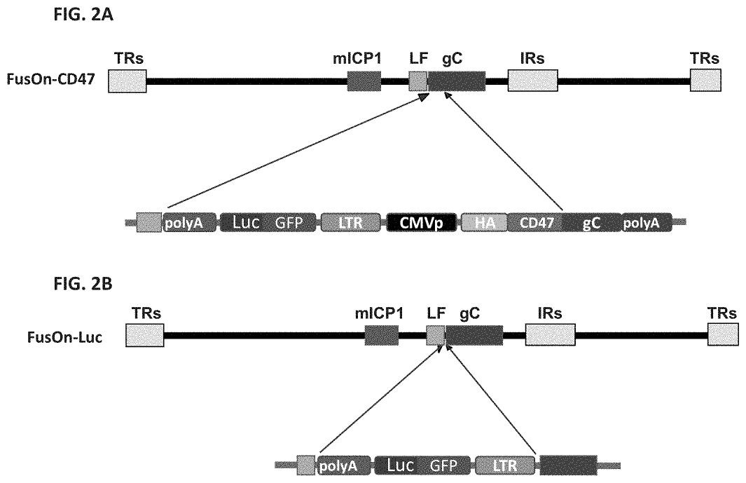

A HA (hemagglutinin) tag was included in cgC to allow the chimeric molecule to be conveniently detected. For the purpose of easiness in identifying the recombinant virus and for in vivo imaging, cgC was linked to another gene cassette that contains the EGFP-luciferase gene. These two gene cassettes were inserted together into the backbone of FusOn-H3, which was derived from a HSV-2 based oncolytic virus (FusOn-H2) by deleting the GFP gene from the virus. FusOn-H2 was constructed by deleting and replacing the N-terminal region of the ICP10 gene with GFP to render it the ability to selectively replicate in and kill tumor cells. The recombinant virus was identified by picking up GFP positive plaques. Each individually picked virus was enriched to homogenous GFP positivity through multiple rounds of plaque purification. The newly generated virus is designated FusOn-CD47-Luc (FIG. 2A). A control virus was also constructed, in which only the EGFP-Luc gene cassette alone was inserted into the backbone of FusOn-H3, FusOn-Luc (FIG. 2B). All the selected viruses maintain the fusogenic property of the parental FusOn-H2.

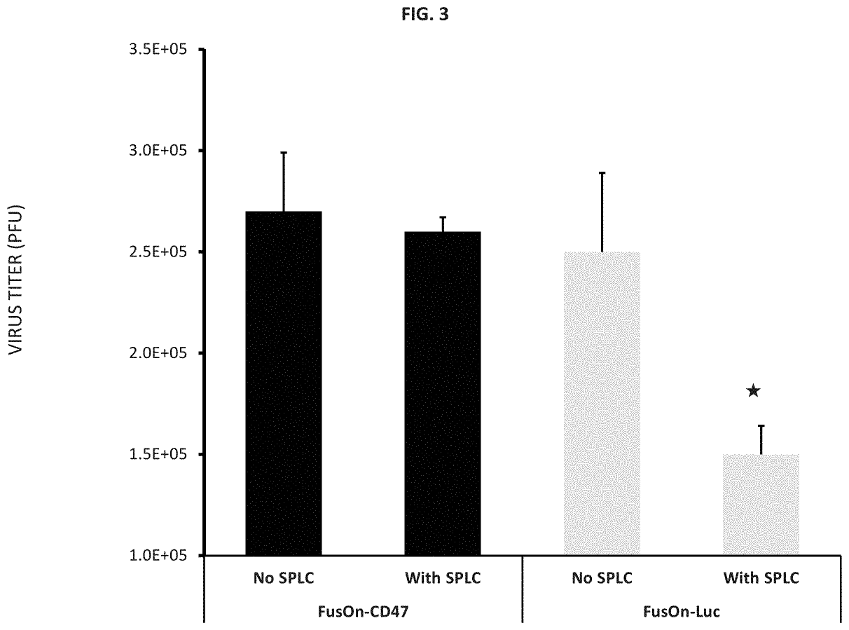

An interesting recent study finds that coating nanoparticles with a CD47 mimetic peptide can help them escape phagocytic clearance by MPS (Rodriguez et al., 2013). The possibility of genetically engrafting the extracellular domain of CD47 molecule to the membrane envelope of an FusOn-H2 was explored, in order to enable it to escape from MPS for systemic delivery. The virus construction strategy is shown in FIG. 2. In vitro assays show that coating the viral particles with CD47 extracellular domain through the recombined gC makes the virus more resistant to the phagocytosis (FIG. 3). When tested in vivo in a murine colon cancer model, the results showed that FusOn-CD47 is 15-fold more effectively than FusOn-Luc to be delivered to the tumor site by the systemic route. Moreover, the data also showed that FusOn-CD47 could persist significantly longer than FusOn-Luc in the tumor site after systemic delivery.

The expression of the transgene by either flow cytometry or western blot analysis was initially examined. For flow cytometry analysis, 293 cells were infected with 1 pfu/cell of either FusOn-CD47-Luc or FusOn-Luc. Twenty-four hours later, cells were labeled with either a PE conjugated anti-mCD47 antibody or a rabbit anti-HA tag antibody. A goat-anti-rabbit antibody conjugated with FITC was added for HA tag detection. Both CD47 and HA tag labelled cells were subjected to flow cytometry analysis. The results in FIG. 2A show that both anti-mCD47 and anti-HA tag antibodies were able to readily detect the cells that had been infected with FusOn-CD47-Luc, but not cells infected with FusOn-Luc. For western blot analysis, 293 cells were either similarly infected with these two viruses or transfected with plasmids that express cgC or wild type gC that does not contain the HA tag. Cell lysates were prepared 24 h later and were subjected to western blot analysis. The result in FIG. 2B shows that cgC is abundantly expressed by FusOn-CD47-Luc but not FusOn-Luc.

Next, quantify of Luc gene expression from these two viruses was compared. Vero, CT26 and 4T1 cells were infected with either FusOn-CD47-Luc or FusOn-Luc at 1 pfu/cell. Cells were harvested 24 h later for measurement of luciferase activity. The result in FIG. 3A showed a near identical level of luciferase activity from cells infected with these two viruses. Another experiment was then conducted to determine, in the in vitro setting, if engrafting an oncolytic HSV with the ECD of mCD47 allows the virus to evade the engulfment and clearance by phagocytes. Mouse splenocytes were used as the source of fresh phagocytes as spleen is the largest unit of the mononuclear phagocyte system. Vero cells were infected with FusOn-CD47-Luc or FusOn-Luc with or without the presence of mouse splenocytes. Cells were collected 48 h later for quantitative measurement of virus yield by plaque assay. The results in FIG. 3B showed that, in the wells without splenocytes, both FusOn-CD47-Luc and FusOn-Luc replicated well and the virus yield was similar in these two wells. However, in the wells with splenocytes, FusOn-Luc yield was significantly reduced while the replication of FusOn-CD47-Luc was only marginally affected. These results indicate that the presence of mCD47 ECD on viral particles enable the virus with the ability to resist the impact from macrophages and possibly some other immune cells during virus infection.

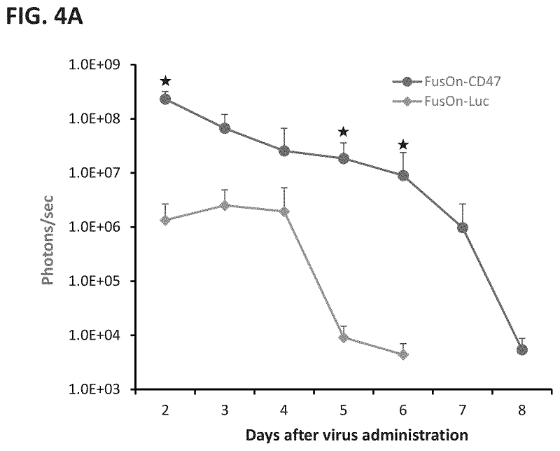

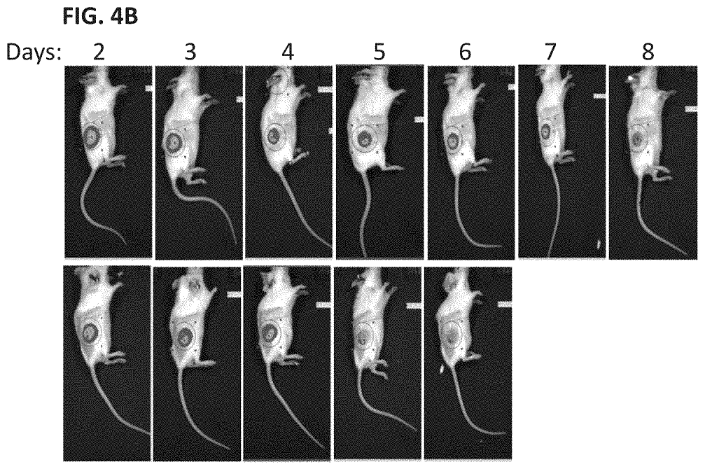

To determine the ability of the incorporated mCD47 ECD in enabling the virus for systemic delivery, CD26 murine colon cancer cells were implanted to the right flank of immunocompetent Balb/c mice. Once tumors reached the approximate size of 8 mm in diameter, we systemically injected 2×106 pfu of either FusOn-CD47-Luc or FusOn-Luc to each mouse. Mice were monitored for luciferase activity by IVIS Spectrum System starting on day 2 and then on a daily basis until total disappearance of the signal. The results in FIG. 4 show that, at day 2 after virus administration, the imaging signal from FusOn-CD47-Luc was approximately one and a half log stronger than that from FusOn-Luc. This indicates that the former was more efficiently delivered to the tumor site than later by the systemic route. Moreover, FusOn-CD47-Luc seems to stay in the tumor tissues substantially longer than FusOn-Luc. By day 5, the imaging signal was barely detectable in tumors from mice receiving FusOn-Luc, while the signal in tumors from FusOn-CD47-Luc remained detectable until day 7. Nevertheless, neither virus showed any significant amplification in tumor tissues because this HSV-2 based oncolytic virus grows poorly in CT26 tumor cells. Interestingly, in one occasion when a few mice were imaged at day 1 after virus delivery, significant imaging signals were detected transiently in the liver. These signals completely disappeared by day 2.

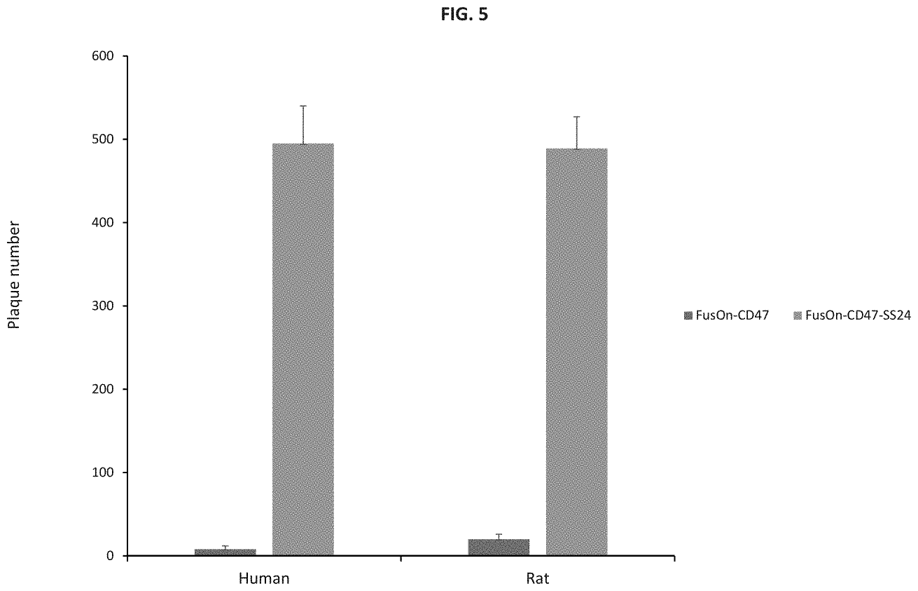

To determine if FusOn-CD47-Luc is also more superior than FusOn-Luc for systemic delivery in other tumor models, the above experiment was repeated, but in mice bearing tumors at the right flank established from implantation of 4T1 murine mammary tumor cells, in which FusOn-H2 was found to be able to grow moderately. Additionally, 4T1 tumor cells secrete macrophage colony stimulating factor (M-CSF) and granulocyte colony stimulating factor (G-CSF), which can enhance macrophage infiltration and phagocytosis. This would allow the CD47-mediated evading strategy to be more vigorously tested. The IVIS imaging results in FIG. 5A indeed showed that the signal in 4T1 tumor was in general lower than those detected in CT26 tumor (as shown in FIG. 4). Nevertheless, the results again showed that FusOn-CD47-Luc could be more efficiently delivered to local tumors than FusOn-Luc by the systemic route. By day 2, difference of the image signal strength between these two viruses was about one and half a log. The biggest difference was recorded on day 4, when the image signal from FusOn-Luc was reduced to nearly background level while it reached the highest for FusOn-CD47-Luc. These data again showed that CD47 modification allows the virus to be more efficiently delivered by the systemic route as well as persisted in tumor tissues longer than the control virus once it had reached there.

To generate a product of FusOn-H2 that can withstand all the three major limiting factors in the bloodstream—the neutralizing antibodies, the complements, and the phagocytes, we subjected FusOn-CD47 to the anti-HSV-2 sera selection as described above. It was subjected to 7 consecutive section with anti-HSV rat sera and then 23 consecutive passage in the presence of rat sera plus one human serum with an extremely high level of anti-HSV-2 antibodies as described above Again, several tests were conducted during the selections to determine if the virus was gaining the ability to resist the neutralizing effect of the immune sera One of the results from such tests is shown in FIG. 5, which was conducted after FusOn-CD47 was first subjected to 7 rounds of section with the rat sera and 17 rounds of section with the rat sera plus the human serum. The results clearly show that, similar to FusOn-SS9, the trained FusOn-CD47 (designated FusOn-CD47-SS24) gained substantial ability in preserving the infectivity in the presence of immune anti-HSV-2 sera.

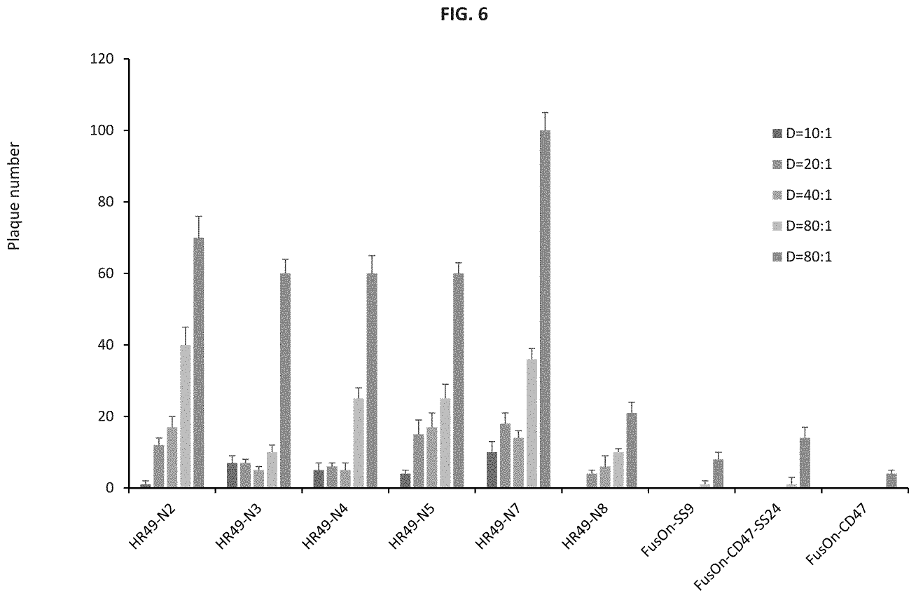

To ensure more profound resistance to the neutralizing antibodies in humans, we subjected the virus to three more rounds of section with a mixture of HSV-2 positive sera obtained from 12 individuals, 4 mixed together a time. The rounds of selection are: 8 for batch 1, 6 for batch 2, and 4 for batch 3. After these extensive selections, the obtained viruses were plaque purified and 6 plaque picks were expanded for further analysis. They are designated HR49-N2-5, HR49-N7 and HR49-N8. First, they were tested on their ability to resist the neutralization from a mixture of eight of the human sera used in the selection, which is the most stringent neutralization test that we have conducted. Both FusOn-SS9 and FusOn-CD47-SS24 were included in this experiment for comparison. The anti-HSV sera mixture was used at different dilutions to incubate with the indicated viruses, after which the virus infectivity was determined. The results in FIG. 6 show that, under this stringent neutralizing condition (containing a mixture of antiviral sera from multiple individuals), the selected virus can still produce a significant number of plaques while the unselected virus did not produce any plaque until the sera mixture was diluted by 80-fold. The data also showed the subsequent three more rounds of selection in the human sera mixtures have further enhanced the ability of the selected virus in their ability to resist the neutralizing effect. Also, the data showed that some of the picked viruses have a better capability than others in resisting the neutralization by the mixed human antiviral sera.

### Example 2

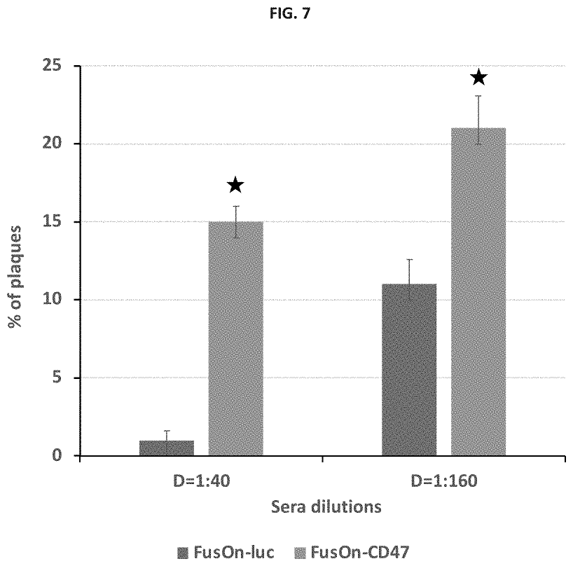

The ability of the oncolytic virus FusOn-H2, with and without insertion of an extracellular domain of CD47 into the N-terminus of gC, to escape neutralization by anti-HSV antibodies was evaluated. In this in vitro experiment, 500 plaque-forming units (pfu) of either FusOn-CD47 or FusOn-Luc were mixed with or without the presence of the diluted anti-HSV-2 sera (at a dilution of 1:40 or 1:160) and incubated at 37° C. before they were applied to Vero cell monolayers for assaying the infectivity and for quantification of plaque formation. The results in FIG. 7 showed that, at 1:40 dilution, the sera could neutralize the virus infectivity of FusOn-Luc almost completely. Even at 1:160 dilution, the sera could still significantly reduce the infection (and hence the number of plaque formation) of FusOn-Luc. In contrast, FusOn-CD47 was much less neutralized in the presence of the same diluted anti-HSV sera. In the presence of 1:40 diluted sera, FusOn-CD47 produced a significant number of plaques (approximately 15% of the well where no anti-HSV sera were added) while FusOn-Luc barely showed any viral plaque at all.

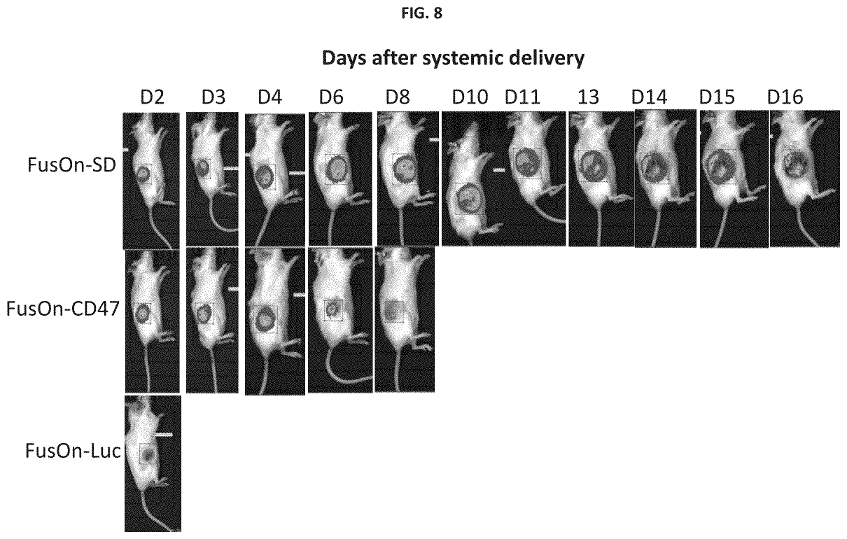

In vivo delivery by the systemic route was evaluated for FusOn-CD47, FusOn-Luc, and FusOn-SD in vaccinated Balb/c mice with HSV-2 and implanted with CT26 murine colon cancer cells in their right flank. 1×10⁷ of either FusOn-CD47, FusOn-Luc, or FusOn-SD was systemically delivered into the mice in different groups by injection via the tail vein. The mice were imaged on the days indicated in FIG. 8 with an IVIS imager. The results showed that FusOn-Luc was unable to be delivered to tumors by the systemic route in the presence of anti-HSV immunity. In contrast, FusOn-CD47 was readily detected in the tumor tissue by the same delivery route and in the presence of anti-HSV immunity. Moreover, FusOn-CD47 persisted in the tumor tissues for almost eight days after reaching the tumor site despite the presence of antiviral immunity. CD47 unexpectedly assisted the oncolytic virus in escaping the neutralization effect by anti-HSV antibodies, contributing to the overall ability of FusOn-SD in escaping neutralizing antibodies for systemic delivery. Accordingly, FusOn-SD behaved the best in systemic delivery to the tumor site. FusOn-SD is thus useful in patients having HSV-1 and/or HSV-2 antibodies, such as those vaccinated against HSV-1 and/or HSV-2 or having HSV-1 and/or HSV-2.

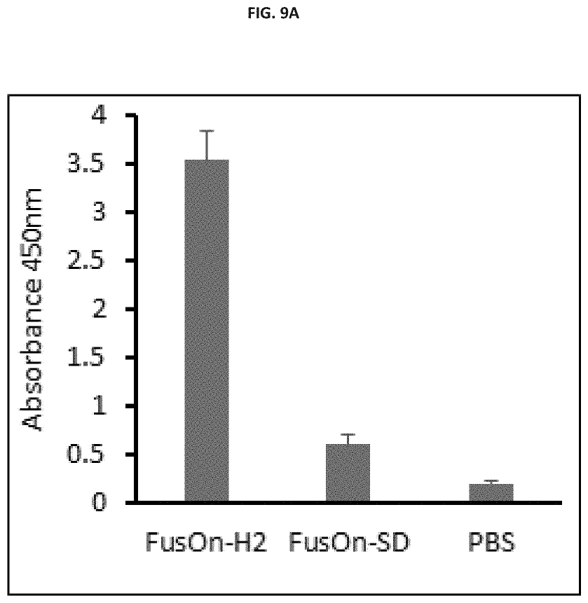

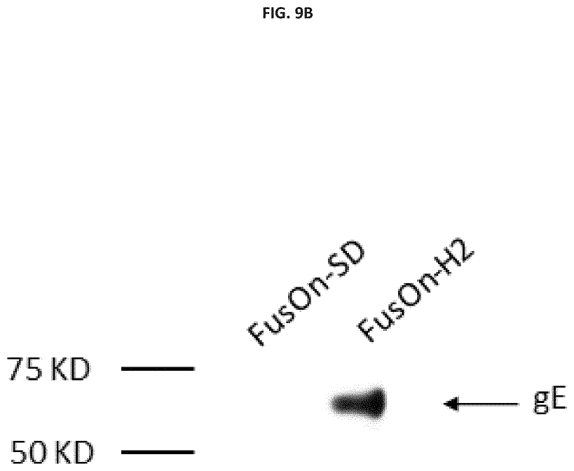

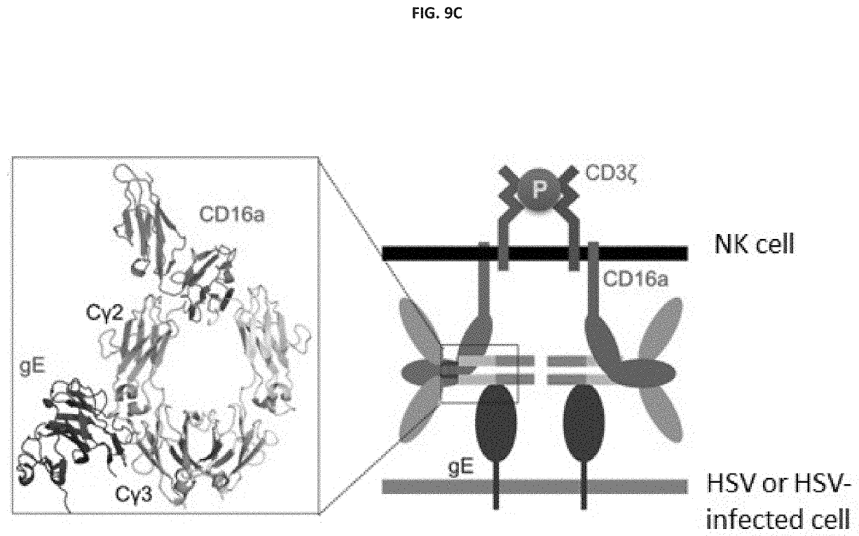

It was unexpectedly found that gE is absent from FusOn-SD viral particles. This is shown by FIGS. 9A to 9C.

FIG. 9A is a bar chart showing the absorbance of FusOn-H2, FusOn-SD, and PBS (negative control) after treatment with mouse anti-HSV-2 gE (1:1000 dilution) and HPR-conjugated rabbit anti-mouse IgG (1:10,000 dilution). More specifically, in FIG. 9A, 1×10⁶ pfu of viruses (the parental FusOn-H2 and FusOn-SD) were coated to 96-well plate for ELISA assay. Wells coated with PBS served as the negative control. The first antibody used was mouse anti-HSV-2 gE (1:1000 dilution) and the second antibody was HPR-conjugated rabbit anti-mouse IgG (1:10,000 dilution). A significant reading was detected in the FusOn-H2, but not in FusOn-SD, indicating that gE was present in FusOn-H2 viral particles, but not in FusOn-SD.

FIG. 9B is a western blot showing detection of gE for FusOn-SD and FusOn-H2. Proteins were subtracted from 1×10⁶ pfu of viruses (FusOn-H2 and FusOn-SD) and were run on an acrylamide gel for western blot analysis. The same antibodies of mouse anti-HSV-2 gE and HPR conjugated rabbit anti-mouse IgG used with regard to FIG. 9A were used for the western blot. The result showed a clear band of gE for FusOn-H2, while it was absent from the lane loaded with proteins from FusOn-SD. This result confirms the finding in FIG. 9A.

FIG. 9C is a schematic illustration on the mechanism of NK cell recognition of HSV or HSV infected cells via gE. The drawing shows that gE binds to IgG (either virus specific or non virus specific). The CD16a, which is one the most important NK cell activation receptors, then binds to the Fc region of the IgG, leading to the activation of NK cells and the clearance of either viral particles or the virus infected cells. This leads to reduced oncolytic virus delivery as well as replication, hence, minimizing the therapeutic effect of virotherapy. Because FusOn-SD is not recognized by this mechanism, NK cells do not clear them out and the therapeutic efficacy of FusOn-SD persists.

### REFERENCES

Bender, F. C., Samanta, M., Heldwein, E. E., de Leon, M. P., Bilman, E., Lou, H., Whitbeck, J. C., Eisenberg, R. J. and Cohen, G. H., 2007. Antigenic and mutational analyses of herpes simplex virus glycoprotein B reveal four functional regions. J Virol 81, 3827-41.
Cairns, T. M., Huang, Z. Y., Gallagher, J. R., Lin, Y., Lou, H., Whitbeck, J. C., Wald, A., Cohen, G. H. and Eisenberg, R. J., 2015. Patient-Specific Neutralizing Antibody Responses to Herpes Simplex Virus Are Attributed to Epitopes on gD, gB, or Both and Can Be Type Specific. J Virol 89, 9213-31.
Cairns, T. M., Huang, Z. Y., Whitbeck, J. C., Ponce de Leon, M., Lou, H., Wald, A., Krummenacher, C., Eisenberg, R. J. and Cohen, G. H., 2014. Dissection of the antibody response against herpes simplex virus glycoproteins in naturally infected humans. J Virol 88, 12612-22.
Eing, B. R., Kuhn, J. E. and Braun, R. W., 1989. Neutralizing activity of antibodies against the major herpes simplex virus type 1 glycoproteins. J Med Virol 27, 59-65.
Ellermann-Eriksen, S., 2005. Macrophages and cytokines in the early defence against herpes simplex virus. Virol J 2, 59.
Fu, X., Tao, L., Li, M., Fisher, W. E. and Zhang, X., 2006. Effective treatment of pancreatic cancer xenografts with a conditionally replicating virus derived from type 2 herpes simplex virus. Clin Cancer Res 12, 3152-3157.
Fu, X. and Zhang, X., 2001. Delivery of herpes simplex virus vectors through liposome formulation. Mol Ther 4, 447-53.
Fulci, G., Dmitrieva, N., Gianni, D., Fontana, E. J., Pan, X., Lu, Y., Kaufman, C. S., Kaur, B., Lawler, S. E., Lee, R. J., Marsh, C. B., Brat, D. J., van Rooijen, N., Stemmer-Rachamimov, A. O., Hochberg, F. H., Weissleder, R., Martuza, R. L. and Chiocca, E. A., 2007. Depletion of peripheral macrophages and brain microglia increases brain tumor titers of persistent viruses. Cancer Res 67, 9398-406.
Greig, S. L., 2016. Talimogene Laherparepvec: First Global Approval. Drugs 76, 147-54.
Huber, V. C., Lynch, J. M., Bucher, D. J., Le, J. and Metzger, D. W., 2001. Fc Receptor-Mediated Phagocytosis Makes a Significant Contribution to Clearance of Influenza Virus Infections. The Journal of Immunology 166, 7381-7388.
Hume, D. A., 2006. The mononuclear phagocyte system. Curr Opin Immunol 18, 49-53.
Kinchen, J. M. and Ravichandran, K. S., 2008. Phagocytic signaling: you can touch, but you can't eat. Curr Biol 18, R521-4.
Macedo, N., Miller, D. M., Haq, R. and Kaufman, H. L., 2020. Clinical landscape of persistent virus research in 2020. Journal for ImmunoTherapy of Cancer 8, e001486.
Nakamori, M., Fu, X., Pettaway, C. A. and Zhang, X., 2004. Potent antitumor activity after systemic delivery of a doubly fusogenic persistent herpes simplex virus against metastatic prostate cancer. Prostate 60, 53-60.
Rodriguez, P. L., Harada, T., Christian, D. A., Pantano, D. A., Tsai, R. K. and Discher, D. E., 2013. Minimal “Self” peptides that inhibit phagocytic clearance and enhance delivery of nanoparticles. Science 339, 971-5.
Silke Heilingloh, C., Lull, C., Kleiser, E., Alt, M., Schipper, L., Witzke, O., Trilling, M., Eis-Hubinger, A. M., Dittmer, U. and Krawczyk, A., 2020. Herpes Simplex Virus Type 2 Is More Difficult to Neutralize by Antibodies Than Herpes Simplex Virus Type 1. Vaccines (Basel) 8.
Tay, M. Z., Wiehe, K. and Pollara, J., 2019. Antibody-Dependent Cellular Phagocytosis in Antiviral Immune Responses. Frontiers in immunology 10.
Tuzmen, C., Cairns, T. M., Atanasiu, D., Lou, H., Saw, W. T., Hall, B. L., Cohen, J. B., Cohen, G. H. and Glorioso, J. C., 2020. Point Mutations in Retargeted gD Eliminate the Sensitivity of EGFR/EGFRvIII-Targeted HSV to Key Neutralizing Antibodies. Molecular Therapy—Methods & Clinical Development 16, 145-154.
Van Strijp, J. A., Van Kessel, K. P., van der Tol, M. E., Fluit, A. C., Snippe, H. and Verhoef, J., 1989. Phagocytosis of herpes simplex virus by human granulocytes and monocytes. Arch Virol 104, 287-98.
Wakimoto, H., Ikeda, K., Abe, T., Ichikawa, T., Hochberg, F. H., Ezekowitz, R. A., Pasternack, M. S. and Chiocca, E. A., 2002. The complement response against an oncolytic virus is species-specific in its activation pathways. Mol Ther 5, 275-82.
Alvarez-Breckenridge, C. A., Yu, J., Price, R., Wei, M., Wang, Y., Nowicki, M. O., Ha, Y. P., Bergin, S., Hwang, C., Fernandez, S. A., Kaur, B., Caligiuri, M. A. and Chiocca, E. A., 2012a. The histone deacetylase inhibitor valproic acid lessens NK cell action against persistent virus-infected glioblastoma cells by inhibition of STAT5/T-BET signaling and generation of gamma interferon. J Virol 86, 4566-77 (“Alvarez-Breckenridge et al., 2012a”).
Alvarez-Breckenridge, C. A., Yu, J., Price, R., Wojton, J., Pradarelli, J., Mao, H., Wei, M., Wang, Y., He, S., Hardcastle, J., Fernandez, S. A., Kaur, B., Lawler, S. E., Vivier, E., Mandelboim, O., Moretta, A., Caligiuri, M. A. and Chiocca, E. A., 2012b. NK cells impede glioblastoma virotherapy through NKp30 and NKp46 natural cytotoxicity receptors. Nat Med 18, 1827-34 (“Alvarez-Breckenridge et al., 2012b”).
Dai, H. S. and Caligiuri, M. A., 2018. Molecular Basis for the Recognition of Herpes Simplex Virus Type 1 Infection by Human Natural Killer Cells. Frontiers in immunology 9, 183-183.
Dubin, G., Basu, S., Mallory, D. L., Basu, M., Tal-Singer, R. and Friedman, H. M., 1994. Characterization of domains of herpes simplex virus type 1 glycoprotein E involved in Fc binding activity for immunoglobulin G aggregates. J Virol 68, 2478-85.
Xie, C. B., Jane-Wit, D. and Pober, J. S., 2020. Complement Membrane Attack Complex: New Roles, Mechanisms of Action, and Therapeutic Targets. The American journal of pathology 190, 1138-1150.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1. A non-replicating herpes simplex virus (HSV) based gene delivery vector, wherein (i) the HSV vector comprises a transgene, and (ii) the HSV vector has a membrane envelope comprising glycoproteins, wherein at least one of the glycoproteins comprises an extracellular CD47 domain inserted into the N-terminus of the glycoprotein.

2. The HSV-based vector of claim 1, wherein the HSV vector is an HSV-1 or HSV-2 vector.

3. The HSV-based vector of claim 1, wherein the HSV-based vector is an HSV amplicon packaged into a viral particle.

4. The HSV-based vector of claim 1, wherein the glycoprotein is selected from glycoprotein C, glycoprotein B, glycoprotein D, glycoprotein H, and glycoprotein L.

5. The HSV-based vector of claim 4, wherein the glycoprotein is glycoprotein C.

6. The HSV-based vector of claim 1, wherein the transgene is a therapeutic gene.

7. The HSV-based vector of claim 1, wherein the extracellular CD47 domain is an extracellular human CD47 domain.

8. A composition comprising the HSV-based vector of claim 1, wherein the HSV vector is prepared by passaging at least twice the HSV vector with immune sera having elevated levels of anti-HSV antibodies.

9. The composition of claim 8, wherein the passaging in the immune sera mutates neutralizing epitopes on glycoprotein B and glycoprotein D of the HSV-based vector.

10. The composition of claim 8, wherein the extracellular CD47 domain comprises amino acids 19-141 of murine CD47.

11. The composition of claim 8, wherein the extracellular CD47 domain is an extracellular human CD47 domain.

12. The composition of claim 8, wherein the HSV-based vector is free or substantially free of gE.

13. The composition of claim 8, wherein the immune sera is a mixture comprising rat sera and human sera that has elevated levels of anti-HSV antibodies.

14. The composition of claim 8, wherein the HSV-based vector has been prepared by passaging at least two times an HSV vector in the presence of rat sera having an elevated level of anti-HSV antibodies followed by passaging at least two times in the presence of a mixture of rat sera and at least one human serum having an elevated level of anti-HSV antibodies.

15. A composition comprising a non-replicating herpes simplex virus (HSV) based gene delivery vector, wherein (i) the HSV-based vector comprises a transgene, and (ii) the HSV-based vector is prepared by passaging at least twice the HSV-based vector with immune sera having elevated levels of anti-HSV antibodies.

16. The composition of claim 15 wherein the HSV-based vector is an HSV-1 or HSV-2 vector.

17. The composition of claim 15, wherein the passaging in the immune sera mutates neutralizing epitopes on glycoprotein B and glycoprotein D of the HSV-based vector.

18. The composition of claim 15, wherein the HSV-based vector is free or substantially free of gE.

19. The composition of claim 15, wherein the immune sera is a mixture comprising rat sera and human sera that has elevated levels of anti-HSV antibodies.

20. The composition of claim 15, wherein the HSV-based vector has been prepared by passaging at least two times the HSV-based vector in the presence of rat sera having an elevated level of anti-HSV antibodies followed by passaging at least two times in the presence of a mixture of rat sera and at least one human serum having an elevated level of anti-HSV antibodies.

21. The composition of claim 15, wherein the transgene is a therapeutic gene.

22. A method of preparing a composition comprising a non-replicating HSV-based vector comprising passaging at least twice an HSV-based vector comprising a transgene with immune sera that has elevated levels of anti-HSV antibodies.

23. The method of claim 22, wherein the HSV-based vector is an HSV-1 or HSV-2 vector.

24. The method of claim 22, wherein the HSV-based vector has a membrane envelope comprising glycoproteins, wherein at least one of the glycoproteins comprises an extracellular CD47 domain inserted into the N-terminus of a glycoprotein.

25. The method of claim 24, wherein the glycoprotein is selected from glycoprotein C, glycoprotein B, glycoprotein D, glycoprotein H, and glycoprotein L.

26. The method of claim 25, wherein the glycoprotein is glycoprotein C.

27. The method of claim 22, wherein the passaging in the immune sera mutates neutralizing epitopes on glycoprotein B and glycoprotein D of the HSV-based vector.

28. The method of claim 24, wherein the extracellular domain comprises amino acids 19-141 of murine CD47.

29. The method of claim 24, wherein the extracellular CD47 domain is an extracellular human CD47 domain.

30. The method of claim 24, wherein the HSV-based vector is free or substantially free of gE.

31. The method of claim 22, wherein the immune sera comprises mammalian anti-HSV antibodies.

32. The method of claim 22, wherein the immune sera is a mixture comprising rat sera and human sera that has elevated levels of anti-HSV antibodies.

33. The method of claim 22, wherein the method comprises passaging the HSV-based vector at least two times in the presence of rat sera having an elevated level of anti-HSV antibodies followed by passaging at least once in the presence of a mixture of rat sera and at least one human serum having an elevated level of anti-HSV antibodies.

34. A method of treating a disease in a patient in need thereof comprising administering to the patient a composition of claim 15.

35. The method of claim 34, wherein the composition or HSV vector is systemically, intramuscularly, intrademally, or subcutaneously administered or administered by intracerebroventricular or intraperitoneal injection.

36. The method of claim 34, wherein the composition comprises cerebrospinal fluid.

37. The method of claim 34, wherein the disease is a neurological disease.

38. The method of claim 34, wherein the patient has been exposed to or vaccinated against HSV-1 and/or HSV-2, or has HSV-1 and/or HSV-2.

39. A method of vaccinating a patient against a malignant or infectious disease comprising administering to the patient a composition of claim 15.
